(19) United States
(12) Patent Application Publication
(10) Pub. No.: US 2014/0276028 A1
(43) Pub. Date: Sep. 18, 2014

(54) Integrated Therapeutic Imaging Catheter and Methods

(71) Applicant: Volcano Corporation, San Diego, CA (US)

(72) Inventors: Jeremy Stigall, Carlsbad, CA (US); Maritess Minas, San Diego, CA (US)

(73) Assignee: Volcano Corporation, San Diego, CA (US)

(21) Appl. No.: 14/212,839

(22) Filed: Mar. 14, 2014

Related U.S. Application Data
(60) Provisional application No. 61/787,065, filed on Mar. 15, 2013.

(51) Int. Cl. A61M 25/01; A61M 25/10; A61B 8/12; A61B 8/08; A61B 5/00

(52) U.S. Cl. CPC A61M 25/0108; A61B 8/0891; A61B 5/0066; A61B 5/0084; A61B 8/12; A61M 25/1006
USPC 600/427; 600/407; 600/463

(57) ABSTRACT

Disclosed herein is an integrated therapeutic and imaging catheter. The catheter comprises an inner member defining a guidewire lumen, a radiopaque balloon assembly, a treatment device operatively associated with an outer sleeve of the balloon assembly, and an imaging device adjacent the balloon assembly. The balloon assembly comprises an inner sleeve surrounding the inner member and an outer sleeve surrounding the inner sleeve. The catheter includes a connection medium, wherein when the connection medium extends through the balloon it is disposed between the balloon inner sleeve and the inner member. The imaging device is disposed adjacent to the balloon assembly and is coupled to the connection medium.

### CROSS-REFERENCE TO RELATED APPLICATIONS

The present application claims priority to and the benefit of U.S. Provisional Patent Application No. 61/787,065, filed Mar. 15, 2013, which is hereby incorporated by reference in its entirety.

### TECHNICAL FIELD

Embodiments of the present disclosure relate generally to the field of medical devices and, more particularly, to integrated therapeutic imaging catheters including a radiopaque balloon assembly.

### BACKGROUND

Intravascular imaging systems are widely used in interventional cardiology as a diagnostic tool for a diseased vessel, such as an artery, within the human body. Various sensors may be placed on a catheter and positioned in the body. One type of imaging system is an intravascular ultrasound (IVUS) system. In one example, a phased array IVUS device includes a number of transducers that are passed into a vessel and guided to an area to be imaged. The transducers emit ultrasonic waves in order to create an image of the vessel of interest. The ultrasonic waves are partially reflected by discontinuities arising from tissue structures (such as the various layers of the vessel wall), red blood cells, and other features of interest. Echoes from the reflected waves are received by the transducer and passed along to an IVUS imaging system. The imaging system processes the received ultrasound echoes to produce a cross-sectional image of the vessel where the device is placed.

Intravascular imaging systems are often used to detect arterial occlusions that can be relieved through use of a balloon catheter. A balloon catheter is a type of catheter with a balloon near the tip. The balloon catheter is designed to be inserted into a patient's artery and positioned to a spot where an occlusion was detected through use of an intravascular imaging system. Upon reaching the detected occlusion, the balloon is inflated to relieve the occlusion. In some instances, the balloon catheter includes a stent, and inflation of the balloon expands and deploys the stent within the vessel.

While existing catheters deliver useful diagnostic imaging information, there is a need for enhanced image quality and ease of use to provide more valuable insight into the condition of vessels and passageways in vivo. Accordingly, there remains a need for improved catheter-type devices, systems, and methods for providing a superior imaging device with clearer images by having fewer and smaller distortions, or speckles, compared to those presently available. Moreover, there is a need for imaging systems that are also capable of treating a patient's vessel in conjunction with monitoring the course of treatment, and for extending the time available to conduct more in-depth imaging or treatment, or both. Thus, an improved catheter is desired to achieve enhanced imaging while also providing a therapeutic effect as disclosed herein.

### SUMMARY

In one aspect, the present disclosure encompasses an integrated therapeutic and imaging catheter that includes an inner member defining a guidewire lumen; a radiopaque balloon assembly that includes an inner sleeve surrounding the inner member, and an outer sleeve surrounding the inner sleeve, wherein the radiopaque balloon assembly is adapted to provide treatment; and an imaging device disposed adjacent to the balloon assembly and coupled to a connection medium.

In a second aspect, the present disclosure encompasses an integrated therapeutic and imaging catheter that includes a radiopaque balloon assembly including an inner balloon sleeve surrounding an inner member, the inner balloon sleeve defining a fluid-tight space therebetween; an imaging device disposed adjacent to the balloon assembly; a treatment device surrounding the balloon assembly; and a connection medium disposed within the catheter and operably connecting the imaging device to a proximal end of the catheter. Various embodiments will now be described that are applicable to any of the aspects described herein.

In one embodiment, at least the inner sleeve or outer sleeve is operatively associated with a radiopaque agent. In a preferred embodiment, the radiopaque agent includes strands, threads, flakes, particles, bands, or a combination thereof, that inhibits or blocks imaging therethrough. In another preferred embodiment, the radiopaque agent includes at least one type of metal blocker sized and shaped sufficiently to inhibit or prevent transmission of imaging waves. In an exemplary embodiment, the metal blocker includes a tungsten doping agent distributed throughout the sleeve.

In another embodiment, the imaging device includes an intravascular ultrasound transducer. In a further embodiment, the imaging device includes an optical coherence tomography device. In one embodiment, the connection medium is disposed between the balloon inner sleeve and the inner member and is configured to move freely within a space therebetween, and in another the inner sleeve is configured to protect the connection medium when the balloon assembly is inflated. In a preferred embodiment, the space defined between the inner sleeve and the inner member includes a fluid.

In further embodiment, the catheter further includes at least one marker band disposed inside the outer sleeve and bonded about the balloon inner sleeve. In another embodiment, two marker bands are disposed at a pre-determined distal distance along the inner sleeve. The marker bands may also be radiopaque, although typically the radiopaque balloon assembly provides this effect without need for the marker bands to also be radiopaque. In another embodiment, an inner lumen disposed inside the inner sleeve and outside the inner member is used to pass a fluid to inflate the balloon assembly. In one embodiment, the fluid may be water, while in another it may be a gas. In a further embodiment, the imaging device is distal to the balloon assembly and the connection medium extends therethrough to the imaging device. In another alternative embodiment, the imaging device is proximal to the balloon assembly.

In an additional embodiment, the outer sleeve is operatively associated with a drug agent for delivery to a patient's vessel. In another embodiment, a therapeutically effective amount of a drug agent is disposed on, or embedded in, the outer sleeve. In one embodiment, the inner sleeve is configured to protect the connection medium when the balloon assembly is inflated at pressures above 20 ATM. In a preferred embodiment, the connection medium includes an electrical conduction wire, an optical fiber, or a combination thereof. In another preferred embodiment, the electrical conduction wire carries data produced by the imaging device. In yet another preferred embodiment, the electrical conduction wire provides power to the imaging device. In a further embodiment, the connection medium includes a driveshaft lumen to drive the imaging device adjacent to the balloon assembly. In another embodiment, the catheter further includes a treatment device that is an expandable stent surrounding the outer sleeve and configured to expand when the balloon assembly is inflated. In another embodiment, the expandable stent is coated in or embedded with a drug agent to provide additional therapeutic treatment. In another embodiment, the inner balloon sleeve is configured to elastically deform inwardly under high operating pressures. In a further embodiment, the inner balloon sleeve is configured to elastically reform to its original shape when the high operating pressures are discontinued.

In one embodiment, the catheter further includes a proximal shaft disposed proximal to the inner balloon sleeve; a distal shaft disposed distal to the inner balloon sleeve, the distal shaft receiving at least a portion of the inner member, the inner balloon sleeve, and the connection medium extending between the inner member and inner balloon sleeve, wherein the inner balloon sleeve is joined to the distal shaft at a distal end of the inner balloon sleeve and is joined to the proximal shaft at a proximal end of the inner balloon sleeve. In another embodiment, the distal shaft includes an independent mid-shaft extending between the sleeve and the imaging device. In a further embodiment, the connection medium is allowed to move freely in the space, which includes a gas. In another embodiment, the proximal shaft includes an axial dual lumen shaft. In one embodiment, the inner sleeve is bonded to an outer lumen of the dual lumen shaft. In another embodiment, an inner lumen of the dual lumen shaft is used to pass an inflation medium to inflate an outer balloon sleeve disposed circumferentially about the inner balloon sleeve.

In a third aspect, the disclosure encompasses a method for diagnosing and treating a patient which includes inserting a catheter into a patient's vessel, the catheter including a radiopaque balloon assembly, a connection medium, and an imaging device into the vessel, wherein the balloon assembly is separated from the imaging device by a first distance and wherein the balloon assembly surrounds the connection medium; imaging a lumen of the vessel with the imaging device; measuring a length of the lesion; moving the catheter based on the length of the lesion to position the radiopaque balloon assembly within the lesion; and treating the lesion while the catheter is in situ. In one embodiment, the method further includes identifying the lesion within the lumen of the vessel with the imaging device.

In one embodiment, at least an inner sleeve or an outer sleeve of the balloon assembly is operatively associated with a radiopaque agent. In a further embodiment, the radiopaque balloon assembly includes an outer sleeve that includes a radiopaque agent. In another embodiment, the radiopaque balloon assembly includes a radiopaque agent disposed and secured about a sleeve of the assembly. In yet another embodiment, the radiopaque agent is selected to include strands, threads, flakes, particles, bands, or a combination thereof, that inhibits or blocks imaging therethrough. In a further embodiment, the radiopaque agent is selected to include at least one type of metal blocker sized and shaped sufficiently to inhibit or prevent transmission of imaging waves. In another embodiment, the metal blocker includes a tungsten doping agent distributed throughout the balloon sleeve.

In one embodiment, imaging the lumen includes imaging while the catheter is advanced through the vessel. In another embodiment, the treating occurs while the catheter maintains a fixed position along the vessel. In a further embodiment, the treating includes inflating the balloon assembly within the lesion using high pressure to compress the lesion against the lumen of the vessel without interfering with the connection medium. In another embodiment, the inflating includes apply a pressure of greater than about 20 ATM to compress the lesion against the lumen of the vessel. In a further embodiment, the treating includes providing a therapeutic agent in operative association with the outer sleeve of the balloon assembly for delivery to a wall of the vessel. In yet another embodiment, which can be alternative or additive to the previous embodiment, the treating includes associating a treatment device with the outer sleeve of the radiopaque balloon assembly, wherein the treatment device is configured to expand with inflation of the balloon assembly. In a preferred embodiment, the treatment device includes an expandable stent, and wherein inflating the balloon assembly within the lesion using high pressure to compress the lesion against the lumen of the vessel includes expanding the expandable stent against the lesion to compress the lesion toward the lumen of the vessel to increase its inner diameter. In a further embodiment, the method further includes deflating the balloon assembly and withdrawing the catheter such that the balloon assembly and the imaging device are positioned proximal to the lesion. In another embodiment, the method further includes imaging the lesion using the imaging device to assess the treatment of the lesion. In yet a further embodiment, the method further includes imaging the stent in an expanded condition using the imaging device to assess the position and expansion of the treatment device and the treatment of the lesion.

### BRIEF DESCRIPTION OF THE DRAWINGS

FIGS. 1A and 1B are diagrams showing illustrative sensing catheters, according to principles described herein.

FIGS. 2A and 2B are diagrams showing an illustrative cross-section taken along line 2-2 of FIGS. 1A and 1B, respectively, of a proximal junction of a balloon catheter, according to embodiments described herein.

FIG. 3 is a diagram showing an illustrative cross-section of a balloon taken along line 3-3 of FIG. 1A according to one embodiment described herein.

FIG. 4 is a diagram showing an illustrative cross-section of a distal junction of a balloon catheter taken along line 4-4 of FIG. 1A according to one embodiment described herein.

FIGS. 5A-5C are diagrams showing an illustrative insertion of a balloon catheter into a patient, according to one example of principles described herein.

FIG. 6 is a flowchart describing an illustrative method for utilizing a therapeutic sensing catheter within a patient, according to one example of principles described herein.

FIG. 7 is a flowchart showing an illustrative method for fabricating a sensing balloon catheter, according to one example of principles described herein.

FIGS. 8A-8F are diagrams showing an illustrative insertion of an integrated catheter into an artery of a patient, according to one example of principles described herein.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications in the described devices, instruments, methods, and any further application of the principles of the disclosure as described herein are contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure.

Embodiments disclosed by the present disclosure are directed to combination catheters that incorporate non-compliant therapeutic devices with imaging systems to accurately access, assess, and treat diseased vessels and/or other tubular structures within a patient. For example, embodiments of the present disclosure are configured to optimize treatment, such as drug delivery and/or stent placement and expansion. The embodiments disclosed herein include balloon stent catheters that incorporate imaging devices such as, by way of non-limiting example, transducers and optical devices operable to perform sensing modalities such as IVUS, optical coherence tomography (OCT), photo acoustic inspection and spectroscopy. In some embodiments, the imaging elements may be oriented generally perpendicular to the axis of the device for side looking imaging while other embodiments may employ axially oriented imaging sensors that provide forward looking imaging ahead of the balloon assembly. The imaging device is disposed adjacent to the radiopaque balloon assembly, and may be distal or proximal thereto as further described herein. Moreover, the embodiments disclosed herein provide a low profile and flexible device that allows for the utilization of high pressure systems with non-compliant therapeutic devices during imaging. Thus, the embodiments disclosed herein allow healthcare professionals to access, assess, and treat intratubular lesions, including arterial and venous lesions, with more ease, less resistance, and more visibility than offered by some conventional catheters.

In an exemplary embodiment of the present disclosure, an intravascular imaging system may be integrated distally along the catheter adjacent to a radiopaque balloon assembly. With such integration, the intravascular imaging system does not have to be first removed from the patient's artery before the balloon can be used to relieve the occlusion. Rather, upon detection of an occlusion, the catheter can be pushed further into the patient or slightly retracted so that the balloon is aligned with the occlusion, and imaging can be conducted all while the catheter is in situ without need to significantly advance or retract the catheter such as to separately provide delivery of the treatment while still providing high quality imaging. In various embodiments, the proximal end of the integrated catheter, typically outside the patient, includes a tri-port adapted to include an inflation port connected to an inflation lumen, a guidewire port (associated with the inner member described herein), and an imaging connector associated with connection media as described further herein.

FIGS. 1A and 1B are diagrams showing illustrative balloon catheter 100 according to certain embodiment of the present disclosure. FIG. 1A illustrates a balloon sensing catheter having a imaging sensor 116 distal to the balloon assembly, while FIG. 1B illustrates a balloon sensing catheter having an imaging sensor 117 proximal to the balloon assembly. In one embodiment, the imaging sensor is electronically activated, and may include, e.g., an IVUS or OCT sensor. In another embodiment, preferably that in FIG. 1B, the imaging sensor may be any suitable sensor including any IVUS or OCT. The IVUS can be a phased-array or a rotational IVUS. The components of the systems have many common elements which will be referred to by the same reference numbers throughout the disclosure. According to certain illustrative examples, the catheter 100 includes a balloon assembly 110 with an outer sleeve 120 and an inner sleeve 108. The balloon assembly 110 is joined to a proximal shaft 104 through a proximal junction 106. Additionally, the balloon assembly 110 is joined to a mid-shaft 114 through a distal junction 112. In the illustrated embodiment of FIG. 1A, the mid-shaft 114 extends between the balloon assembly 110 and a sensing device 116. In the embodiment of FIG. 1B, the sensing device 117 is proximal to the balloon assembly, such that the mid-shaft 114 may be shortened relative to the other embodiment (or in an embodiment not depicted, eliminated) and adjacent the distal tip 118 of the catheter, and may be tapered to a tip if desired (not shown). An inner member 102 defining a guide wire lumen 103 can run from the tip 118 of the catheter, through the interior of the proximal shaft 104, the balloon assembly 110, and the mid-shaft 114, to at least the proximal end of the balloon assembly 110.

The proximal shaft 104 connects the balloon assembly 110 to a pressurized fluid system while a connection medium 208 (FIGS. 2A, 2B), such as a microcable, electrical conductors, or optical fibers, extending within the proximal shaft connect the sensing device 116 or 117 to a processing system (not shown) at the proximal end of the catheter 100. In one aspect, the sensing device 116 is an ultrasound transducer array having a maximum outer diameter sized to fit within the mid-shaft 114 (e.g., about 3.5 F) and the connection medium 208 is a microcable having a braided exterior with 7 individual insulated electrical conductors. In another aspect, the connection medium 208 includes fiberoptics, such as for an OCT sensor. In some embodiments, such as shown in FIG. 1A, the connection medium 208 extends through the entire length of the balloon assembly 110 and joins the sensing device 116. In other embodiments, the connection medium 208 extends to the sensing device 117 but does not need to pass through the balloon assembly since the assembly is distal to the sensing device 117 in FIG. 1B. The processing systems typically remain outside of the patient at a proximal end of the catheter. The processing system uses the data received from the sensing device 116 or 117. When the sensing device 116 or 117 is part of an imaging system, the data can be used to create an image. The image can be displayed to a medical professional in real-time as the catheter moves through the patient's artery, vein, or other suitable vessel. This allows the medical professional to find various occlusions or other irregularities that may exist throughout the patient's vessel. In a similar manner, the sensing device 116 or 117 could be a pressure or flow sensor, and the processing system could determine fractional flow reserve values based on the sensed data.

The proximal shaft may be made of any suitable material, e.g., a surgically acceptable plastic, polymer, metal, or other flexible material, or any combination thereof. In one aspect, the proximal shaft may include a metal proximal portion joined to a distal polymer tube with a metal wire embedded in the polymer tubing adjacent the coupling to transition the stiffness of the tubing from the stiffer metal to the more flexible polymer tubing. The proximal shaft 104 is designed to be flexible so that it may effectively traverse a patient's vessel without damaging the structure, e.g., of a vein or artery. The proximal shaft 104 may be a dual lumen shaft. The dual lumen proximal shaft 104 may be an axial dual lumen shaft with an inner lumen and an outer lumen.

The proximal shaft 104 may have a diameter within the range of about 2 to 4 French (i.e., 0.67 to 1.33 mm). The length of the proximal shaft 104 is long enough to allow the balloon 110 and the sensing device 116 or 117 to reach a sufficiently deep region of a patient's vessel. For example, the proximal shaft 104 may have a length of approximately 150 cm. In a collapsed condition, the maximum outer diameter of the balloon assembly is approximately 0.04 inches.

The inner member 102 defines a guidewire lumen 103 that is sized to receive a guide-wire (shown in various figures including FIGS. 2A and 2B). In one embodiment, the guidewire lumen has a diameter of about 0.017 inches, such that it can receive an about 0.014 inch diameter guidewire. In use, a guide-wire may be first inserted into a patient's vessel. The catheter may then be placed over the guide-wire such that the inner member 102 encompasses the guide-wire. In some examples, the inner member 102 may extend the entire length of the catheter 100, from the tip 118 to the proximal end of the proximal shaft 104. Such a catheter is referred to as an over-the-wire catheter. In some examples, the inner member 102 may extend along a short distance and then exit out of the catheter at an exit port near the proximal end of the balloon 110. Such a catheter is referred to as a rapid-exchange catheter.

The length of the inner member is long enough to extend from the point at which the catheter starts on the guide-wire (typically, the tip 118) to the point at which the guide-wire exits the catheter. Thus, the length may be relatively short in the case of a rapid-exchange catheter and relatively longer in the case of an over-the-wire catheter.

The mid-shaft 114 is connected between the distal end of the balloon 110 and the sensing device 116, or between the proximal end of the balloon 110 and sensing device 117. The mid-shaft 114 is typically made of a polymer, plastic, or other flexible material, or a combination thereof. The same material or an indpendently selected material may be used to form the proximal shaft 104. The mid-shaft 114 is flexible so that it may effectively traverse a patient's vessel without damage. The inner member 102 runs through the interior of the mid-shaft 114. Additionally, a connection medium 208 runs from the sensing device 116 towards the balloon 110 through the mid-shaft 114 in the embodiment of FIG. 1A, and from the sensing device 117 towards a proximal end of the catheter without passing through the balloon assembly 110 in the embodiment of FIG. 1B.

In one embodiment, one, two, or more marker bands (not shown) may be included in the balloon assembly to show the location and length of the balloon assembly when deployed. The marker band(s) are preferably disposed inside the outer sleeve and bonded about the balloon inner sleeve. These are disposed at a pre-determined distal distance along the inner sleeve to facilitate use of the integrated therapeutic imaging catheter. They can be selected of any suitable material, but in various embodiments they are radiopaque, or opaque to the selected imaging sensor(s) 116 or 117 provided in the catheter device disclosed herein, or both.

FIG. 2A is a diagram showing an illustrative cross-section of a proximal junction 106 of the balloon catheter 100 according to one embodiment of the present disclosure. The proximal junction 106 connects the proximal end of the balloon to the proximal shaft (e.g., 104, FIG. 1A). According to certain illustrative examples, the proximal shaft is a dual lumen shaft that includes an inner lumen 204 and an outer lumen 202. The proximal junction 106 also includes the inner member 102, the inner balloon sleeve 108, and a space through which connection media 208 run. In an unshown embodiment, the connection media 208 may be secured in a separate lumen in this space. The proximal junction 106 further includes a balloon proximal leg 206. In one aspect, the balloon proximal leg 206 is an extension of the material forming the balloon outer sleeve 120.

FIG. 2B illustrates a cross-sectional view of the embodiment shown in FIG. 1B. The embodiment of FIG. 2B includes no connection media 208 because the imaging sensor 117 is on the proximal side of the balloon assembly and cross-section of FIG. 2B. In this embodiment, a rotational imaging sensor, phased-array sensor, or any suitable imaging sensor device may be used. Thus, the connection media 208 could include a rotary drive cable assembly, and the cable could include an outer sheath surrounding an inner drive cable and a series of electrical conductors or optical fibers.

The outer lumen 202 of the proximal shaft 104 provides an external structure for the proximal shaft 104. The inner lumen 204 is smaller in diameter than the outer lumen 202 and runs axially within the outer lumen 202. The size of the inner lumen 204 is such that there is sufficient room within the outer lumen for the inner member 102, inner balloon sleeve 108, and in FIG. 2A, connection media 208.

The inner lumen 204 can be used to pump inflation fluid into the balloon. Thus, the end of the inner lumen 204 within the proximal junction 106 serves as an inflation port where the inflation fluid exits the inner lumen 204 into the balloon. The inflation fluid exits into the space between the balloon inner sleeve 108 and the balloon outer sleeve, thus inflating the balloon. Any suitable fluid may be used, including those that conventionally caused speckling and other imaging distortion, because the balloon assembly, e.g., the inner sleeve 108, the outer sleeve 120, or both, is radiopaque. This can permit selection from a wide array of different fluids based on, e.g., patient safety, viscosity, conductivity, and other surgical considerations beyond simply providing for superior image quality. Typically, however, the radiopaque agent is intended to block X-rays so convention saline fluid may be desired in various embodiments.

A radiopaque balloon assembly 110 is formed to include at least one surface, such as the inner balloon sleeve 108 or outer balloon sleeve 120 to be radiopaque. This can be achieved in any suitable way, including coating or disposing a radiopaque agent over the surface, or embedding, distributing, composing, or blending a radiopaque agent in the surface. In an exemplary embodiment, the radiopaque agent is associated with the inner balloon sleeve 108. In another, it is associated with the outer balloon sleeve 120. X-ray blocking material is preferred as the radiopaque agent. The radiopaque agent in various embodiments may include strands, threads, flakes, particles, bands, or a combination thereof, that inhibits or blocks imaging therethrough. Preferably, the radiopaque agent comprises at least one type of metal blocker sized and shaped sufficiently to inhibit or prevent transmission of imaging waves. The metal blocker in various exemplary embodiments may include tungsten, gold, iron, platinum, barium, bismuth, or the like, or any combination, blend, or alloy thereof. Iodine may also be used as the radiopaque agent. An exemplary embodiment involves a tungsten doping agent distributed throughout the balloon sleeve to provide a radiopaque effect.

The sleeve that is operatively associated with the radiopaque agent may include the radiopaque agent on either or both sides of the selected balloon sleeve 108, 120. In some applications, a portion less than all of the radiopaque balloon 110 can be provided with radiopaque properties rather than the entire balloon 110. Where partial area radiopaque properties are utilized in fabricating radiopaque balloon assembly 110, the determination of the balloon shape during X-ray fluoroscopy may be an important consideration.

The balloon inner sleeve 108 acts as a barrier between the inflation fluid and any structures that run through the internal portion of the catheter, particularly, any connection media 208 as well as the inner member 102. The balloon inner sleeve 108 is bonded to the interior of the outer lumen 202 of the proximal shaft 104. Additionally the balloon inner sleeve 108 encompasses the inner member 102. As shown more fully in FIG. 3, the balloon inner sleeve 108 is sized such that there is a sufficient space 212 between the sleeve 108 and the inner member 102 so as to allow any connection media 208 to fit therein. This space 212 allows the connection media 208 to float freely without damaging the integrity of the balloon. However, bonding material 213 can be used to fill the space in the proximal connection 106 and distal connection 112 to define the fluid tight region 212 within inner sleeve 108 beneath balloon 120.

In one aspect, the inner sleeve 108 is formed of a multi-layer structure suitable for high pressure operation greater than 20 atmospheres (ATM). In some embodiments, the inner sleeve 108 is configured to be suitable for operating pressures extending through, by way of example only, a range of about 15 to 25 ATM. In one aspect, this range may include about 17 to 22 ATM. In another aspect, this range may include about 19 to 21 ATM. Other ranges are contemplated. The material properties and construction of the inner sleeve 108 typically allow it to deform under high pressure without significant elongation along the longitudinal axis of the balloon assembly, even under application of the contemplated high pressures. In some embodiments, the materials forming the inner sleeve 108 permit very little, if any, axial compression and extension, even under the application of high pressures.

The balloon assembly 110 may be formed of any conventionally suitable material, so long as it is further made radiopaque as described herein. In one embodiment, the inner sleeve is formed by an inner layer of polyethylene (PE) bonded to an outer layer of maleated polyethylene. The outer layer of maleated PE is more suitable for heat-treated bonding to other components of the system, such as the proximal shaft 104 and mid-shaft 114, that can be formed of PEBAX polymer. Other suitable balloon assembly materials include one or more nylon polymers, PET polymers, Kevlar® material, and combinations of any of these balloon assembly materials. It will be understood that the proximal shaft 104, the mid-shaft 114, and the inner shaft 102 are formed such that they do not deform under high operating pressures while the inner sleeve 108 is designed to intentionally elastically deform inwardly under the high operating pressures of the balloon system. The inner sleeve 108 is shaped and configured to collapse around the connection media 208 or inner shaft 102 without damaging or otherwise interfering with the operation of the connection media or other structure running through the inner sleeve 108. The inner sleeve 108 material and configuration are selected so it will elastically return to its original shape when the high pressure condition is removed. Return of the inner sleeve 108 to its original shape may also be aided by compressed gas within the space 212.

Various types of connection media may run through the proximal shaft 104 to the imaging sensor 116 or 117. In FIG. 1B, this can be any suitable connection media for any suitable imaging sensor 117. In FIG. 1A, space considerations inside the balloon cause the fixed sensor types to be preferred. Thus, various figures include space 212 between the inner member 102 and the balloon inner sleeve 108 to include desired connection media 208. For example, in the case that the sensing device produces electrical signals to be processed by external systems, then the connection media 208 may include conductive wires to carry those electrical signals. Alternatively, the connection media may include fiber optic cables to propagate those signals in the form of light. The number of wires or cables depends on the type of sensing device and the manner in which data is transferred from the sensing device to the external processing systems. Conductive wires may also be used to provide electrical power to the sensing device. These are not depicted in FIG. 2B because this is proximal to the imaging sensor 117 discussed above.

In the case that the imaging sensor 117 is rotational, the connection media 208 may include a driveshaft lumen. In one aspect, the driveshaft lumen may include a plastic sheath filled with a liquid lubricant. The lubricant allows the driveshaft running through the plastic sheath to spin with a minimal amount of friction against the interior of the plastic sheath.

The balloon proximal leg 206 is part of the balloon outer sleeve (e.g., 120, FIG. 1A). The balloon proximal leg 206 is designed to fit securely around the exterior of the distal shaft. The balloon proximal leg 206 may be bonded to the exterior of the distal shaft through a variety of bonding methods. These bonding methods include, but are not limited to, thermal bonding and laser bonding.

FIG. 3 is a diagram showing an illustrative cross-section of the balloon assembly 110 taken along line 3-3 of FIG. 1A. According to certain illustrative examples, the cross-section includes the balloon outer sleeve 120, the balloon inner sleeve 108, the connection media 208, and the inner member 102. The diameter of the balloon depends on the amount of inflation fluid 302 pumped into the balloon through the proximal junction. For non-distensible balloon materials, the balloon diameter is fixed to a specific diameter. In one embodiment, the non-compliant balloon has a working length of approximately 15 mm and is available in expanded diameters ranging from about 2 mm to 4 mm in roughly 0.5 mm increments. In one embodiment, the outer diameter of the balloon assembly in the collapsed state is approximately 0.040 inches.

The proximal shaft 104 at the proximal end of the balloon and the mid-shaft 114 at the distal end of the balloon are typically independent shafts. According to certain illustrative examples, there is not a continuous shaft extending through the interior of the balloon. Rather, the interior of the balloon includes only the connection media 208 in certain embodiments with the distal imaging sensor 116 as in FIG. 1A, and the inner member 102. This provides additional flexibility within the balloon, particularly the embodiment of FIG. 1B that does not even include connection media through the balloon assembly 110. Moreover, this allows the connection media 208 of FIG. 1A to float freely within the space 212 between the balloon inner sleeve 108 and the inner member 102. In the illustrated example of FIG. 2A, the ends of the balloon inner sleeve 108 are sealed to the respective proximal and distal catheter components forming the fluid tight chamber 212 surrounding microcable 208 and inner member 102. In some cases, as shown in FIG. 3, the space 212 may be filled with air or other gases, while in some cases the space 212 may be filled with a liquid.

As mentioned above, an inflation fluid is used to inflate the balloon when it is appropriately aligned in order to perform various medical tasks such as relieving an arterial occlusion. Thus, the diameter of the balloon outer sleeve 120 changes based on the inflation status of the balloon. As the balloon is non-compliant, the diameter only extends to a certain point. The non-compliant nature of the balloon prevents too much expansion within a patient's vein or other vessel. The balloon inner sleeve 108 is designed with integrity such that the balloon inner sleeve 108 will not place too great of a pressure on any connection media 208 present when the balloon is inflated (as in the embodiment of FIGS. 1A and 2A).

FIG. 4 is a diagram showing an illustrative cross-section of FIG. 1A of the distal junction 112 of the balloon catheter 100 according to one embodiment of the present disclosure. According to certain illustrative examples, the distal junction 112 connects the balloon to the mid-shaft 114 at the distal end of the balloon. The distal junction 112 includes the inner member 102, the inner balloon sleeve 108, and the space 212 through which the connection media 208 runs. The distal junction 112 further includes a balloon distal leg 402.

The mid-shaft 114 is an independent shaft that is connected adjacent its proximal end to the distal end of the balloon and either the tip 118 in FIG. 1B or in FIG. 1A, adjacent its distal end to the sensing device 116. The mid-shaft 114 is also designed to be flexible in order to allow the catheter to effectively traverse a patient's vessel. The mid-shaft 114 may have a diameter within the range of about 2.5 to 4 French (i.e., 0.83 to 1.33 mm).

The length of the mid-shaft 114 depends on the desired distance between the distal end of the balloon and either the sensing device 116 or the tip (FIGS. 1A, 1B, respectively). The length may be long enough so that the sensing device 116 does not interfere with the distal junction as the catheter traverses sharper turns. The length of the mid-shaft may also be short enough so as not to push the sensing device too much deeper into the patient's vessel when using the balloon to relieve a vascular occlusion. In one example, the length of the mid-shaft has a length of about 3 mm to 15 mm with an exemplary length of about 5 mm to 10 mm.

The distal end of balloon inner sleeve 108 is typically bonded to the interior of the mid-shaft 114. Additionally, the exterior of the mid-shaft 114 is bonded to the balloon distal leg 402. The balloon distal leg 402 is part of the balloon outer sleeve and is designed to fit securely around the mid-shaft 114. In one preferred embodiment where the mid-shaft is independent from the proximal shaft, the integrated catheter has an overall greater flexibility. Additionally, the connection media 208, when included as in the embodiment of FIGS. 1A and 2A, are allowed to float freely through the center of the balloon without including the integrity of the balloon. In one aspect, the connection medium 208 includes a braided microcable having three to twenty, preferably seven, individually insulated electrical conductors. In the illustrated embodiment of FIG. 3, the external braid material has been removed so that each conductor can float independently within the space 212 defined within inner sleeve 108. It will be appreciated that the during the bonding process, the individual conductors will have some slack between the distal and proximal bonding areas such that the conductors can be curved to follow tortuous vessel paths and can migrate over one another under high pressure balloon inflation. The relatively free movement of the conductors within the balloon assembly increases the low profile and highly flexible assembly that inhibits conductor breakage. This is particularly helpful when the catheter herein is used to provide a fluid-tight inflation system for high pressure capabilities above 20 ATM.

As mentioned above, the balloon assembly 110 can be used to relieve various types of vascular occlusions. When the balloon assembly 110 is appropriately positioned within a patient's vessel, the balloon outer sleeve 120 is then inflated to put pressure on the occlusion. The balloon outer sleeve 120 is typically inflated with an inflation fluid. The inflation fluid is typically a saline fluid, as such a fluid is harmless to the patient if it leaks into the artery. The inflation fluid may be pumped into the balloon through an inner lumen of the proximal shaft 104 to a range of 15 to 20 ATM, or even greater depending on material properties of the balloon.

According to certain illustrative examples, the balloon outer sleeve 120 is a non-compliant balloon. A non-compliant balloon is one that is designed to inflate to a particular diameter and not stretch beyond that diameter. This prevents the balloon outer sleeve 120 from expanding too much. This is important because excess expansion could damage a patient's artery or other vessel. The balloon outer sleeve 120 may also be designed to resist too much axial compression, which could allow a non-compliant balloon outer sleeve 120 to expand farther than desired. Additionally, the balloon outer sleeve 120 may be designed to resist too much axial stretching, which could prevent the balloon outer sleeve 120 from expanding to the desired diameter. In some embodiments, as detailed below in FIGS. 8A-8F, a stent can be positioned in a compressed state around the balloon for delivery to a site of stenosis. The balloon may be inflated to plastically expand the stent to open the vessel and the stent can remain in a supporting position after the balloon is deflated.

As mentioned above, the sensing device 116 or 117 can be used to image the interior of a patient's vessel. Various types of sensing devices may be used. One example of a sensing device 116 or 117 is an OCT device. In another form, the sensor can collect information for spectroscopy or photo acoustic imaging. The sensing device 116 may also be a forward looking device that scans forward into the vessel rather than outward from the axis towards the vessel walls.

The sensing device 116 or 117 may also be an IVUS device. There are two general types of IVUS devices that may be used. The first type of device is a solid state device, also known as a phased array. This is preferred in various embodiments for the imaging sensor 116 or 117, but particularly in the embodiment of FIG. 1A. Solid-state IVUS devices carry a transducer complex that includes an array of ultrasound transducers distributed around the circumference of the device. The transducers are connected to a set of transducer controllers. The transducer controllers select individual transducers for transmitting an ultrasound pulse and for receiving the echo signal. By stepping through a sequence of transmit-receive pairs, the solid-state IVUS system can synthesize the effect of a mechanically scanned transducer element, but without moving parts. Because there is no rotating mechanical element, the transducer array can be placed in direct contact with the blood and vessel tissue with minimal risk of vessel trauma. Furthermore, the interface is simplified because there is no rotating element. The solid-state scanner can be wired directly to the imaging system with a simple electrical cable and a standard detachable electrical connector.

In the example of a transducer array as a sensing device, the connection medium 208 running through the catheter shafts includes the electrical cables that communicate data between the transducer array and external processing systems. The number of wires and cables including the connection media may depend on the type of transducer array. For example, a 64-bit array may use more cables than a 32-bit array. Additionally, various multiplexing functions may be used to reduce the number of connection media 208 (e.g., wires) running through the catheter.

The second general type of IVUS device is a rotational device. A typical rotational IVUS device includes a single ultrasound transducer element located at the tip of a flexible driveshaft. This type of IVUS, if used, can more readily be the imaging sensor 117 of FIGS. 1A and 2A because no driveshaft need pass through the balloon assembly 110. The transducer can be any suitable one, such as a traditional planar PZT type transducer or a focused transducer such as a PMUT type device that permits Focused Acoustic Computed Tomography (FACT). In one aspect, the transducer is positioned distally of the balloon while in another embodiment the transducer is positioned within the inner sleeve 108 within the balloon assembly. The driveshaft spins inside a plastic sheath inserted into the vessel of interest. The transducer element is oriented such that the ultrasound beam propagates generally perpendicular to the axis of the device. The fluid-filled sheath protects the vessel tissue from the spinning transducer and driveshaft while permitting ultrasound signals to propagate from the transducer into the tissue and back. As the driveshaft rotates, the transducer is periodically excited with a high voltage pulse to emit a short burst of ultrasound. The same transducer then listens for the returning echoes reflected from various tissue structures. The IVUS imaging system assembles a two dimensional display of the vessel cross-section from a sequence of pulse/acquisition cycles occurring during a single revolution of the transducer.

In the example of a rotational array as the sensing device 117, the connection media running through the catheter shafts includes a driveshaft lumen that includes a plastic sheath surrounding a driveshaft used to drive the rotational array. Additionally, the connection media include any electrical cables that communicate data between the transducer array and external processing systems.

FIGS. 5A-5C are diagrams showing an illustrative insertion of a balloon catheter into a patient. The present invention can be used in a variety of lumens, vessels or passages in the body including, but not limited to, arteries such as coronary, carotid or peripheral, veins, structural heart, digestive system, organs and brain. According to certain illustrative examples, a guide-wire 506 is fed into a patient's vessel 504. In one aspect, a guidewire having a diameter of approximately 0.014 inches can be utilized. The catheter can then be moved along that guide-wire 506 deeper into the patient's vessel 504.

FIG. 5A is a diagram 500 showing an integrated catheter being pushed into a patient's vessel 504. The tip of the catheter 502 can be designed to facilitate such entry. Although not shown, it will be understood that in some applications a guiding catheter having a minimum internal diameter of approximately 6 French (i.e., 0.066 inches or 2 mm) may be used to facilitate placement of the sensing balloon catheter. At this point, the balloon is not inflated. The catheter is pushed into the vessel 504 until the distal junction of the balloon enters the vessel 504. The catheter 502 is then pushed further into until the proximal junction enters the vessel 504. Thereafter, the catheter 502 is pushed further into the vessel with the proximal shaft 512 extending outside the vessel 504 and outside the patient.

FIG. 5B is a diagram 510 showing the catheter 502 moving through the patient's vessel. According to certain illustrative examples, the catheter 502 traverses the vessel 504 as a doctor views the data obtained by the sensing device. This data will inform the doctor if there is some type of occlusion 508. Upon finding such an occlusion 508, the catheter 502 is pushed further into, or retracted away from, the patient a known distance such that the balloon is aligned with the occlusion 508. Particularly, using an integrated sensor 117 in FIG. 1B, may permit retraction of the catheter to position the balloon assembly, which can minimize trauma or damage when extending anything further proximally into a patient's vessel 504.

FIG. 5C is a diagram 520 showing the integrated balloon catheter 502 inflated in order to relieve an arterial or venous occlusion. According to certain illustrative examples, upon being appropriately aligned, the balloon is inflated to relieve the occlusion. As mentioned above, this is done by pumping an inflation fluid through an inner lumen of the proximal shaft 512. As the proximal shaft 512 is flexible, it bends appropriately in order to enter and traverse the vessel 504 without causing damage.

FIG. 6 is a flowchart showing an illustrative method 600 for inserting a radiopaque balloon catheter into a patient. According to certain illustrative examples, the method includes inserting 602 a tip 118 of a catheter into a patient, the catheter designed to follow a guide-wire, the tip 118 including a sensing device. The method further includes continuing 604 to insert the catheter into the patient along the guide-wire so that a distal end of a radiopaque balloon enters the patient, a junction at the distal end including an inner member, a balloon inner sleeve encompassing the inner member and bonded to an interior of a mid-shaft, and a balloon distal leg bonded to an exterior of the mid-shaft. The method further includes continuing 606 to insert the catheter into the patient along the guide-wire so that a proximal end of the balloon enters the patient, a junction at the proximal end that includes a proximal shaft, an interior of the proximal shaft bonded to the balloon inner sleeve, and a balloon proximal leg bonded to an exterior of the proximal shaft, the connection medium being disposed between the balloon inner sleeve and the inner member. In another embodiment (not shown), the sensing device 117 may be proximal to the radiopaque balloon assembly. In this embodiment, connection media do not necessarily extend into or through the radiopaque balloon assembly.

FIG. 7 is a flowchart showing an illustrative method for fabricating a radiopaque balloon catheter. According to certain illustrative examples, the method includes bonding 702 a distal end of a balloon inner sleeve to an interior of a mid-shaft, the balloon inner sleeve encompassing an inner member. The method further includes bonding 704 a proximal end of the balloon inner sleeve to a proximal shaft, and routing 706 a connection medium between a space between said balloon inner sleeve and said inner member.

FIGS. 8A-8E illustrate the insertion of an integrated therapeutic and imaging catheter or integrated catheter 800 into a patient. The integrated catheter 800 includes a balloon assembly 802 and an imaging device 803, which are substantially similar to the balloon assembly 110 and the sensing device 116, respectively, except for any differences noted herein. The inner sleeve 804 of the integrated catheter 800 is substantially similar to the inner sleeve 108 except for any differences noted herein. As mentioned above in relation to the inner sleeve 108, in some embodiments, the inner sleeve 804 has high pressure capability greater than 20 , ATM which makes the balloon assembly 802 suitable for non-compliant post dilatation. For example, FIGS. 8A-8E illustrate the use of the integrated catheter 800 to access an intravascular lesion 806, assess the intravascular lesion, and treat the intravascular lesion using a treatment device, such as an expandable stent 808, optionally with a drug-eluting coating thereon. In another embodiment (not shown), no stent 808 is used, and the balloon assembly 802 is coated or embedded with a therapeutic drug in an therapeutically effective amount that contacts an inner wall of the patient's vessel for release primarily after the balloon is expanded to contact its outer surface with the inner wall, according to various embodiments of the present disclosure.

In the pictured embodiment, the treatment device includes the expandable stent 808. In other embodiments, the treatment device may include any of a variety of expandable devices shaped and configured to be carried on the balloon assembly 802 for the treatment of intratubular lesions, e.g., intravascular lesions, any of which may include or be coated or embedded with a therapeutic drug in a therapeutically effective amount. For example, the treatment device may include a scaffolding device, a valve device, a filtering device, a stent graft, a sensor device, an ablation device, or a drug delivery or elution device. In some instances, the treatment device may include a resorbable device, such as, by way of non-limiting example, a resorbable stent. In some instances, the treatment device may be designed to indefinitely remain in the vessel after removable of the catheter 800. In other instances, the treatment device may be designed for removal along with the catheter 800 or removal at a later time.

FIG. 8A illustrates the integrated catheter 800 being advanced into a patient's vessel, e.g., a vein or artery 810. Initially, a guide-wire 812 is fed into the vessel 810. In one aspect, a guidewire having a diameter of approximately 0.014 inches can be utilized. The catheter can then be moved along the guide-wire 802 deeper into the patient's vessel 504. During insertion of the catheter 800 into the vessel 810, the balloon assembly 802 is not inflated and maintains a low profile in an unexpanded condition. A distal end 814 of the catheter 800 can be designed to facilitate entry and progress through the vessel 810. For example, the distal end 814 may be tapered at or adjacent the tip.

As shown in FIG. 8A, the catheter 800 is pushed into the vessel 810 until the imaging device 803 and a distal junction 816 of the balloon assembly 802 enters the vessel 810. The catheter 800 is then pushed further into the vessel 810 until a proximal junction 818 of the balloon assembly 802 enters the vessel 810. Thereafter, the catheter 800 is pushed further into the vessel 810 with a proximal shaft 820 extending outside the vessel 810 and outside the patient.

FIG. 8B illustrates the catheter 800 moving through the lesion 806 in the patient's vessel 810. The imaging device 803 can be used to detect and assess the lesion 806. It should be understood that this embodiment employs the arrangement of FIGS. 1A and 2A where the imaging sensor 116 is distal to the balloon assembly, but that in operation the arrangement of FIGS. 1B and 2B could be used where the imaging sensor 117 is proximal to the balloon assembly. The lesion 806 includes a proximal end 825 and a distal end 830, as well as a length L1 extending from the proximal end 825 to the distal end 830. As the catheter 502 traverses the vessel 810, a healthcare professional can view the data obtained by the imaging device 803 to assess the health of the vessel. The imaging data can inform the doctor if there is some type of intravascular lesion or injury, such as, by way of non-limiting example, the intravascular lesion 806. The imaging data may also relay other vascular characteristics, such as, by way of non-limiting example, the path and/or tortuosity of the vessel 810, the regularity or irregularity of the vessel walls within the vessel 810, and various characteristics about the blood flow within the vessel 810. Upon visualizing the lesion 806, the catheter 800 is advanced further into the vessel 810 until the balloon assembly 802 is aligned with the occlusion 806. The imaging device 803 can continue to image the vessel as the distal end 814 of the catheter 800 travels through the lesion 806, thereby providing the healthcare professional with an accurate assessment of the location of the balloon assembly 802. In particular, the imaging device 803 is positioned a known distance D1 from the balloon assembly 802, which allows a healthcare professional to advance and/or retract the catheter 800 the known distance to position the balloon assembly 802 relative to whatever intravascular position the imaging device 803 is imaging at a given time. A corresponding procedure to all those described herein can be used when the integrated catheter includes imaging sensor 117, dependent on the type of imaging sensor selected.

The imaging device 803 can also be used to facilitate placement of the balloon assembly 802 relative to the lesion 806. In the illustrated example, the lesion 806 is an intravascular occlusion that requires reduction and stenting as treatment. As shown in FIGS. 8B and 8C, as the imaging device 803 travels through the lesion, the image data relayed by the imaging device 803 can inform the healthcare professional of various anatomic characteristics within the vessel 810, such as, by way of non-limiting example, the length L1 of the lesion 806, the luminal contours of the lesion 806 (e.g., the intraluminal diameter of the artery 810 proximal, adjacent, and distal to the lesion 806), and characteristics of the blood flow through the lesion 806. Using this imaging data, the healthcare professional can advance the catheter 800 an appropriate distance forward to accurately position the unexpanded balloon assembly 802 and optional but depicted overlying stent 808 within the lesion 806. The stent 808 includes a length L2 extending from a proximal stent end 835 to a distal stent end 840. The healthcare professional can assess whether the length L2 of the stent is appropriate to treat the lesion 806, which has the length L1. In addition, the healthcare professional may verify that the diameter of the stent is appropriate to treat the lesion 806. If the stent 808 is comparatively too short, too long, too wide, or too slender to appropriately treat the lesion 806, the catheter 800 may be removed and replaced with a catheter carrying a correctly-sized stent, thereby avoiding the potential stent failure or collapse that may accompany implantation of an inappropriately-sized stent.

FIG. 8C illustrates the expansion of the balloon assembly 802 and the stent 808 within the lesion 806 in the patient's vessel 810. After the healthcare professional advances the balloon assembly 820 and the stent 808 (in an unexpanded condition) appropriately within the lesion 806, the healthcare professional may inflate the balloon assembly 802 to both relive the occlusion caused by the lesion 806 and expand the stent 808 to maintain the new patency of the vessel 810 at the location of the lesion 806. As mentioned above, this may be done by pumping an inflation fluid through an inner lumen of the proximal shaft 820 of the catheter 800. As the balloon assembly 802 is inflated under a high pressure, typically in the range of about 15-25 ATM, the stent 808 assumes an expanded condition and flattens the lesion 806 against inner walls of the vessel 810.

FIG. 8D illustrates the withdrawal of the balloon assembly 802 from the lesion 806 after initial deployment of the stent 808 within the lesion 806. The healthcare professional may deflate the balloon assembly 802 and retract the catheter 800 until the imaging device 803 is positioned proximal to the stent 808. The healthcare professional can use imaging data received by the imaging device 803, now positioned proximal to the lesion 806 and the stent 808, to assess the expansion and deployment of the stent 808. In particular, the imaging data allows the healthcare professional to verify appropriate stent apposition against the lesion 806 and expansion within the vessel 810. Occasionally, as shown in FIG. 8D, the expansion of the stent 808 is insufficient to adequately treat the lesion 806. For example, in the pictured embodiment, the stent 808 has not fully expanded to compress the lesion 806 against luminal walls 845 of the vessel 810. Instead, the lesion 806 remains partially intact and capable of at least partially occluding flow through the vessel 810. The imaging device 803 can convey this information via imaging data to the healthcare professional.

FIG. 8E illustrates the reinsertion and re-expansion of the balloon assembly 802 within the lesion 806. After assessing the stent deployment, if the healthcare professional desires to increase the expansion of the stent 808 and further decrease the profile of the lesion 806, the healthcare professional may re-advance the catheter 800 and re-position the balloon assembly within the stent 808 and the lesion 806. As shown in FIG. 8E, the balloon assembly 802 may be re-inflated at a higher pressure, which may compress the lesion or, when present in the previous discussion, further expand the stent 808, thereby expanding the luminal walls 845 of the vessel 810 and/or improving the stent apposition to expand the walls 845.

For example, if the initial inflation pressure was 17 ATM, the subsequent inflation pressure may be 20 ATM. In another example, if the initial inflation pressure was 20 ATM, the subsequent inflation pressure may be 25 ATM. Other changes in pressure between the initial and subsequent pressure are contemplated. In some embodiments, the subsequent pressure may be greater than the initial pressure by a predetermined percentage. For example, in one instance, the subsequent inflation pressure may be at least about 25% greater than the initial inflation pressure. Other predetermined percentage increases are contemplated, such as a 20% or 40% increase in inflation pressure. In some embodiments, the healthcare provider may select the change or delta between the initial pressure and the subsequent pressure depending upon the desired degree of further expansion of the treatment device.

FIG. 8F illustrates the withdrawal of the balloon assembly 802 from the lesion 806 after the secondary expansion of the stent 808 within the lesion 806. The healthcare professional may once again deflate the balloon assembly 802 and retract the catheter 800 until the imaging device 803 is positioned proximal to the stent 808. The healthcare professional can use imaging data received by the imaging device 803 to assess the expansion and deployment of the stent 808. In particular, the imaging data allows the healthcare professional to verify appropriate stent apposition against the lesion 806 and expansion within the vessel 810. If the imaging data indicates appropriate deployment of the stent 808 (i.e., appropriate positioning, expansion, and apposition), then the healthcare professional may withdraw the catheter 800 from the vessel 810 (and the patient's body).

In another embodiment, a patient can have a vessel, such as a vein or artery, both diagnosed and treated with the integrated catheter remaining in situ in the vessel without removal from the patient, which can minimize or avoid complications. The integrated catheter disclosed herein can be inserted into a patient's vessel, a lumen of the vessel can be imaged with the imaging sensor, and moving the catheter (e.g., based on the length of the lesion when measured) to position the radiopaque balloon assembly within the lesion. Then, the method includes treating the lesion while the catheter is in situ. In one embodiment, the lesion may be identified and/or characterized, or optionally a length of the lesion is measured to facilitate treatment through best possible positioning of the catheter. Any suitable radiopaque agent as discussed herein can be used.

The imaging the lumen can be achieved while the catheter is advanced through the vessel, while it is stationary, or at discrete points during advancement such as pausing the advancement to increase the image quality when imaging. The treating typically occurs while the catheter maintains a fixed position along the vessel.

The treating is typically achieved by inflating the balloon assembly within the lesion using high pressure to compress the lesion against the lumen of the vessel, which can occur without interfering with the connection medium present in certain embodiments. The inflating includes applying a pressure of greater than about 20 ATM to compress the lesion against the lumen of the vessel, or any other suitable pressure described herein.

In some embodiments, the treating includes providing a therapeutic agent, such as a drug, in operative association with the outer sleeve of the balloon assembly or a treatment device associated with the balloon assembly for delivery to an inner wall of the vessel being imaged and optionally, but preferably, treated. The treating may include associating a treatment device with the outer sleeve of the radiopaque balloon assembly, wherein the treatment device is configured to expand with inflation of the balloon assembly. This can include an expandable stent, and then inflating the balloon assembly within the lesion using high pressure compresses the lesion against the lumen of the vessel by expanding the expandable stent against the lesion to compress the lesion toward the lumen of the vessel to increase its inner diameter. The stent can be coated with, embedded with, or otherwise associated with a therapeutic agent such as any convention drug-eluting material. Alternatively, the outer balloon sleeve may be coated with, embedded with, or otherwise associated with such a therapeutic agent. Of course, both techniques may be used, to administer different forms of the same therapeutic agent or different therapeutic agents.

The treatment method can further includes deflating the balloon assembly and either withdrawing the catheter or advancing the catheter such that the balloon assembly and the imaging device are positioned proximal to the lesion. The lesion or expanded stent may then be imaged using the imaging device to assess the position and expansion of the treatment device and the treatment of the lesion, whereupon a further treatment can be administered as discussed herein as needed. For example, where a therapeutic agent is embedded in the outer sleeve of the balloon, it may be administered only when the balloon is expanded so the sleeve contacts the inner lumen of the vessel, so additional treatment time with the expanded balloon may provide further treatment.

In another embodiment, the catheter may include a balloon assembly, an imaging device, and an ablation device. In other embodiments, the catheter may include a balloon assembly, an imaging device, and an electrical stimulation device. In some embodiments, these treatment devices could be used to denervate target tissue. As described above with reference to FIGS. 8A-8F, the healthcare professional may inflate the balloon assembly at increasingly higher pressures in combination with imaging to verify the accurate positioning, repositioning, and real-time use of these treatment devices.

The integrated catheter and methods disclosed herein are well suited for percutaneous transluminal angioplasty (PTA), including the radiopaque balloon assembly that minimizes and typically avoids the need for contrast medium in such procedures such as during X-ray spectroscopy. The integrated catheter and methods are adapted to treat patients with renal, iliac, femoral, popliteal, tibial, peroneal, and subclavian arteries and other vascular vessels to treat obstructive lesions thereof, such as native synesthetic arteriovenous dialysis fistulae and other venous applications. Imaging and/or balloon inflation (or dilation) may take place before or after treatment, such as with a stent or drug agent. Such images can aid in assessment and/or documentation of the treatment results.

Although illustrative embodiments have been shown and described, a wide range of modification, change, and substitution is contemplated in the foregoing disclosure and in some instances, some features of the present disclosure may be employed without a corresponding use of the other features. It is understood that such variations may be made in the foregoing without departing from the scope of the present disclosure. Accordingly, it is appropriate that the appended claims be construed broadly and in a manner consistent with the scope of the present disclosure.

What is claimed is:

1. An integrated therapeutic and imaging catheter, comprising:
an inner member defining a guidewire lumen;
a radiopaque balloon assembly comprising:
an inner sleeve surrounding the inner member, and
an outer sleeve surrounding the inner sleeve;
wherein the radiopaque balloon assembly is adapted to provide treatment; and
an imaging device disposed adjacent to the balloon assembly and coupled to a connection medium.

2. The catheter of claim 1, wherein at least the inner sleeve or outer sleeve is operatively associated with a radiopaque agent.

3. The catheter of claim 2, wherein the radiopaque agent comprises strands, threads, flakes, particles, bands, or a combination thereof, that inhibits or blocks imaging therethrough.

4. The catheter of claim 3, wherein the radiopaque agent comprises at least one type of metal blocker sized and shaped sufficiently to inhibit or prevent transmission of imaging waves.

5. The catheter of claim 4, wherein the metal blocker comprises a tungsten doping agent distributed throughout the outer sleeve.

6. The catheter of claim 1, wherein the imaging device comprises an intravascular ultrasound transducer or an optical coherence tomography device.

7. The catheter of claim 1, wherein the connection medium is disposed between the balloon inner sleeve and the inner member and is configured to move freely within a space therebetween, and wherein the inner sleeve is configured to protect the connection medium when the balloon assembly is inflated.

8. The catheter of claim 7, wherein the space defined between the inner sleeve and the inner member comprises a fluid.

9. The catheter of claim 1, further comprising at least one marker band disposed inside the outer sleeve and bonded about the balloon inner sleeve.

10. The catheter of claim 9, wherein two marker bands are disposed at a pre-determined distal distance along the inner sleeve.

11. The catheter of claim 1, wherein an inner lumen disposed inside the inner sleeve and outside the inner member is used to pass a fluid to inflate the balloon assembly.

12. The catheter of claim 1, wherein the inner sleeve is configured to protect the connection medium when the balloon assembly is inflated at pressures above 20 ATM.

13. The catheter of claim 1, wherein the inner balloon sleeve is configured to elastically deform inwardly under high operating pressures.

14. The catheter of claim 13, wherein the inner balloon sleeve is configured to elastically reform to its original shape when the high operating pressures are discontinued.

15. An integrated therapeutic and imaging catheter, comprising:
a radiopaque balloon assembly comprising an inner balloon sleeve surrounding an inner member, the inner balloon sleeve defining a fluid-tight space therebetween;
an imaging device disposed adjacent to the balloon assembly;
a treatment device surrounding the balloon assembly; and
a connection medium disposed within the catheter and operably connecting the imaging device to a proximal end of the catheter.

16. The catheter of claim 15, further comprising:
a proximal shaft disposed proximal to the inner balloon sleeve;
a distal shaft disposed distal to the inner balloon sleeve, the distal shaft receiving at least a portion of the inner member, the inner balloon sleeve, and the connection medium extending between the inner member and inner balloon sleeve, wherein the inner balloon sleeve is joined to the distal shaft at a distal end of the inner balloon sleeve and is joined to the proximal shaft at a proximal end of the inner balloon sleeve.

17. The catheter of claim 15, wherein the distal shaft comprises an independent mid-shaft extending between the sleeve and the imaging device.

18. The catheter of claim 15, wherein the connection medium is allowed to move freely in the space, which includes a gas.

19. The catheter of claim 15, wherein the proximal shaft comprises an axial dual lumen shaft.

20. The catheter of claim 15, wherein the inner sleeve is bonded to an outer lumen of the dual lumen shaft.

21. The catheter of claim 15, wherein an inner lumen of the dual lumen shaft is used to pass an inflation medium to inflate an outer balloon sleeve disposed circumferentially about the inner balloon sleeve.

22. The catheter of claim 15, wherein the inner balloon sleeve is configured to elastically deform inwardly under high operating pressures to protect the connection medium when the balloon assembly is inflated.

23. The catheter of claim 22, wherein the inner balloon sleeve is configured to elastically reform to its original shape when the high operating pressures are discontinued.

24. The catheter of claim 15, wherein at least the inner sleeve or the outer sleeve is operatively associated with a radiopaque agent.

25. The catheter of claim 24, wherein the radiopaque agent comprises strands, threads, flakes, particles, bands, or a combination thereof, that inhibits or blocks imaging therethrough.
